(19) United States
(12) Patent Application Publication
(10) Pub. No.: US 2021/0379326 A1
(43) Pub. Date: Dec. 9, 2021

(54) Mood Adjuster Device and Methods of Use

(71) Applicant: Adrian Pelkus, Lexington, SC (US)

(72) Inventor: Adrian Pelkus, Lexington, SC (US)

(21) Appl. No.: 16/873,070

(22) PCT Filed: Jul. 31, 2018
(86) PCT No.: PCT/US18/44462

§ 371 Date: Jan. 27, 2020

Related U.S. Application Data
(60) Provisional application No. 62/538,870, filed on Jul. 31, 2017.

(51) Int. Cl. A61M 21/00; G16H 10/60; G16H 20/30; G16H 20/70; G16H 40/63; G16H 50/20; A61M 21/02; G06F 3/01

(52) U.S. Cl. CPC A61M 21/0094; A61M 2230/50; G16H 20/30; G16H 20/70; G16H 40/63; G16H 50/20; A61M 21/02; G06F 3/011; A61M 2021/0016; A61M 2021/0022; A61M 2021/0066; A61M 2205/054; A61M 2205/3553; A61M 2205/80; A61M 2230/06; A61M 2230/42; G16H 10/60

(57) ABSTRACT

The mood adjuster device comprises: a housing; a plurality of detectors or sensors for obtaining information on the physical characteristics of an individual; a plurality of sensory stimulus generators for providing sensory stimulus to the individual; a controller that receives information from the detectors or sensors and analyzes the information to determine a physical treatment; a memory for storing the information received by the plurality of detectors or sensors, the analyzes performed by the controller and the physical treatment as applied by the sensory stimulus generators; a user interface to allow the individual to communicate with the controller; a medical practitioner interface for accessing information stored in the memory or for providing instructions to the controller or to interface directly with the individual; and a power supply.

### CROSS-REFERENCE TO RELATED APPLICATIONS

This application claims the benefit of the filing date of PCT/US2018/44462 filed 31 Jul. 2018 under 35 U.S.C. § 371 and the filing date of provisional patent application Ser. No. 62/538,870 filed Jul. 31, 2017 from which the PCT application claims priority.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATION-BY-REFERENCE OF MATERIAL SUBMITTED ON A COMPACT DISC

Not applicable

### TECHNICAL FIELD

The present invention relates generally to devices for and methods of adjusting an individual's emotional state or mood. More specifically, the invention provides apparatus and methods for diagnosing and providing therapies to alter an individual's emotional states. The device obtains pertinent physical information from an individual, analyzes the information and applies certain sensory stimulus that may be tactile, visual, olfactory or auditory, to treat the individual who may be suffering from anxiety, sadness or other undesirable mental state.

### BACKGROUND OF THE INVENTION

A wide variety of devices have been constructed that attempt to affect the emotional state or mood of the individual in need of such care. However, none have attempted to integrate information obtained from a variety of physical states of the individual, assess the existing condition as compared to a baseline created for individuals of similar characteristic as the present individual or based on a baseline of that individual and then treat the individual utilizing a full complement of sensory inputs of the individual including visual, sound, smell and tactile stimulus. As such, there is a continuing unmet need for a device and methods of using the device to achieve an emotional state or mood adjustment when an individual is faced with an emotional state that could compromise that particular individual and those around them.

Various limitations of the related art will become apparent to those skilled in the art upon a reading and understanding of the specification below and the accompanying drawings.

### SUMMARY OF THE INVENTION

The device herein disclosed and described is a device for adjusting an individual's emotional state or mood comprising: a housing; a plurality of detectors or sensors for obtaining information on the physical characteristics of the individual; a plurality of sensory stimulus generators for providing sensory stimulus to the individual; a controller that receives information from the detectors or sensors and analyzes the information to determine a physical treatment; a memory for storing the information received by the plurality of detectors or sensors, the analyzes performed by the controller and the physical treatment as applied by the sensory stimulus generators; a user interface to allow the individual to communicate with the controller; a medical practitioner interface for accessing information stored in the memory or for providing instructions to the controller or to interface directly with the individual and a power supply. Each of these elements is connected to the controller and either affixed within the housing or connected to the housing. The treatment determined for the individual either by the controller or the medical practitioner is administered through the plurality of sensory stimulus generators.

In one embodiment of this aspect of the invention, the housing may be a visor or helmet or similar head gear, a desk top mounted device, a free standing device, a partial body chamber or a full body chamber. Further, the device may have radio frequency shielded housing and the power supply may be earth grounded.

In another embodiment of the present invention the device may further comprise a CO₂ detector and oxygen concentrator, wherein the atmosphere local to the individual being treated is monitored for its CO₂ content and that content may be diluted or adjusted with oxygen to lower the CO₂ ppm.

In another embodiment, there are at least three of the detectors or sensors that may be a combination of any of a brain wave monitor, an iris scanner, a facial recognition system or camera, a respiratory monitor, a temperature sensor, a heart rate or pulse rate monitor, a CO₂ detector and a voice recognition system.

In yet another embodiment, there are at least two sensory stimulus generators that may be a combination of PEMF coils, a virtual reality image screen, an aroma generator, a temperature controller, a tens generator or a tactile feedback system.

In still another embodiment, the user interface may be a written communication system or a verbal communication system. There may also be a medical practitioner interface that allows the practitioner to obtain information from the device, communicate and/or direct the controller or communicate directly with the individual. This communication may be a wireless connection.

In still another embodiment, the power supply is a rechargeable battery and may be replaceably affixed within the housing.

Another aspect of the present invention is a method of adjusting an individual's emotional state or mood comprising the steps of: having the individual desiring adjustment of their emotional state or mood interface with the device above; obtaining information on the physical characteristics of the individual from the plurality of detectors or sensors; analyzing the information to determine a physical treatment for the individual; instituting the treatment by providing sensory stimulus through the sensory stimulus generators, thereby adjusting the emotional state or mood of the individual; and storing the information of the physical characteristics, analyses and physical treatment of the individual.

In one embodiment of this aspect of the invention a method of adjusting an individual's emotional state or mood comprising the steps of: having said individual desiring adjustment of their emotional state or mood interface with a device for adjusting an individual's emotional state or mood, wherein said device comprises: an atmospherically controlled housing; a plurality of detectors or sensors for obtaining information on the physical characteristics of said individual affixed within said atmospherically controlled housing; a plurality of sensory stimulus generators for providing sensory stimulus to said individual connected to said atmospherically controlled housing; a controller that receives information from said plurality of detectors or sensors and analyzes said information to determine a physical treatment, wherein said plurality of sensory stimulus generators are connected to said controller and wherein said physical treatment is provided by one or more of said plurality of sensory stimulus generators, wherein said controller is affixed within said atmospherically controlled housing; a memory connected to said controller for storing said information received by said plurality of detectors or sensors, said analyzes performed by said controller and said physical treatment as applied by said sensory stimulus generators as directed by said controller, wherein said memory is affixed within said atmospherically controlled housing; a user interface connected to said controller to allow said individual to communicate with said controller, wherein said user interface is connected to said atmospherically controlled housing; and a housing, wherein said housing is radio frequency shielded (e.g., Faraday shield) and atmospherically controlled, and an earth grounded power supply connected to said controller obtaining information on the physical characteristics of said individual from said plurality of detectors or sensors; analyzing said information to determine a physical treatment for said individual; instituting said treatment by providing sensory stimulus through said sensory stimulus generators, thereby adjusting the emotional state or mood of said individual; and storing said information of said physical characteristics, analyses and physical treatment of said individual.

In other embodiments, the methods may provide the device further comprising a CO₂ detector and/or an oxygen concentrator.

With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention.

The objects, features, and advantages of the invention will be brought out in the following part of the specification, wherein detailed description is for the purpose of fully disclosing the invention without placing limitations thereon.

### BRIEF DESCRIPTION OF THE DRAWINGS

Understanding of the present invention will be facilitated by consideration of the following detailed description of a preferred embodiment of the present invention, taken in conjunction with the accompanying drawings, in which like reference numerals refer to like parts and in which;

FIG. 1 is a method flow chart depicting one embodiment of the present invention's process for personal identification, to systematically diagnose an individual while simultaneously generating treatments or therapies, measuring the individual's biologic reactions to the treatment and/or therapies, to store all data collected or created and to communicate with the individual as well as other medical practitioners;

FIG. 2A is a block diagram of one embodiment of the present invention and 2B is a block diagram of a second embodiment of the present invention;

FIG. 3A is a block diagram of one embodiment of the device's inputs and 3B is a block diagram of a second embodiment of the device's inputs;

FIG. 4A is a block diagram of one embodiment of the device's outputs and 4B is a block diagram of a second embodiment of the device's outputs;

FIG. 5A are schematic electric circuit block diagrams of the input circuits of one embodiment of the present invention and 5B are schematic electric circuit block diagrams of the input circuits of a second embodiment of the present invention;

FIG. 6A are schematic electric circuit block diagrams of the output circuits of one embodiment of the present invention and 6B are schematic electric circuit block diagrams of the output circuits of a second embodiment of the present invention; and

FIG. 7 shows 3 possible configurations of the device of the present invention.

### DETAILED DESCRIPTION OF THE INVENTION

Unless defined otherwise, all terms used herein have the same meaning as are commonly understood by one of skill in the art to which this invention belongs. All patents, patent applications and publications referred to throughout the disclosure herein are incorporated by reference in their entirety. In the event that there is a plurality of definitions for a term herein, those in this section prevail.

The term “connected” as used herein refers to the physical joining of one element of the invention to another element of the invention as well as the electrical connectivity of one electrical component of the invention with another electrical component of the invention. This term includes affixing by adhesive, by screw and bolt, by screw alone, by rivet or other similar method known to those skilled in the art. This also includes electronic connectivity wherein one electrical component is connected to another electrical component by an electrically conductive wire or by a wireless connection.

The phrase “atmosphere local to the individual being treated” may refer to the area around the nose and mouth of the subject using the device, such as a visor that covers the nose and/or mouth of the user, an area around the head of the subject using the device, such as a helmet providing an enclosure around the head of the user, a partial or small chamber surrounding and enclosing the upper torso of the user or a chamber in which the user resides during treatment. These areas may be fully enclosed or partially enclosed and are provided to enable adjustment of the gas (e.g., CO₂, O₂, etc.) concentration and content about the user to effect treatment.

The present invention is a device and a method of using the device to systematically identify and diagnose an individual while simultaneously measuring the biologic reactions of that individual to prescribed therapies or treatments required to relieve them of stress, anxiety and other negative or undesirable emotional states. The emotional or mood adjusting device is able to diagnose, deliver therapies or treatments, measure reactions or responses to those therapies or treatments as well as communicate and entertain the individual through an integrated apparatus comprised of multiple sensors including audio, temperature, respiratory rate, heart rate, iris motion detection, facial recognition and brainwave activity as it's inputs. In response, the device generates tactile feedback, temperature control, tens signals, audio and speech, aromatherapy, atmospheric modification, PEMF and virtual reality visual screen outputs to provide stimulus to the individual's respiratory and audio, visual, olfactory and tactile sensory inputs of their brain ultimately effecting their brainwaves, blood chemistry and subsequently their moods, emotions and attitudes.

The present invention encompasses a device for adjusting the emotional state or mood of an individual as well as methods of using the device to cause an emotional state or mood adjustment in an individual. The device comprises: a housing; a plurality of detectors or sensors affixed within the housing for obtaining information on the physical characteristics of the individual; a plurality of sensory stimulus generators connected to the housing for providing sensory stimulus to the individual; a controller that receives information from the plurality of detectors or sensors and analyzes the information to determine a physical treatment, wherein the plurality of sensory stimulus generators are connected to the controller and wherein the physical treatment is provided by one or more of the plurality of sensory stimulus generators connected to the controller, wherein said controller is affixed within the housing; a memory connected to the controller for storing the information received by the plurality of detectors or sensors, the analyzes performed by the controller and the physical treatment as applied by the sensory stimulus generators as directed by the controller, wherein said memory is affixed within the housing; a user interface connected to the controller to allow the individual to communicate with the controller, wherein the user interface is connected to the housing; a medical practitioner interface connected to the controller for accessing information stored in the memory or for providing instructions to the controller or to interface directly with the individual; and a power supply connected to the controller.

In operation the present invention can perform a variety of functions including detecting, monitoring, analyzing and recording brain waves, scanning and analyzing the iris, scanning and analyzing facial features of the user, measuring, monitoring and recording respiratory rate, measuring and recording temperatures, measuring, monitoring and recording heart or pulse rate as well as recognizing, monitoring, analyzing and recording the voice to determine if the individual, such as an astronaut's, emotional conditions in comparison to baseline measurements are at acceptable levels.

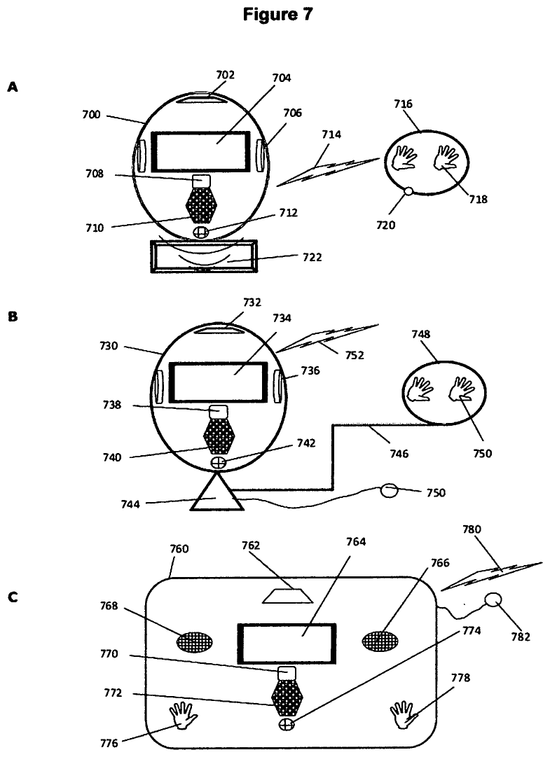

The invention may be provided in a variety of configurations such as a visor or helmet connected to a table top or stand-alone control unit (FIG. 7 A). It may include a small chamber for the head or upper torso connected to or integrated into a control unit or it may be an enclosure in which the control units is integrated into one of the walls of the enclosure. It may be constructed as a kiosk (FIG. 7 B) or as a mirror or wall mounted framed device (Figure C). In one embodiment, the invention is prepared in the form of a helmet 700 and a handheld orb 716 that is held by the user (FIG. 7). The helmet comprises an energy source and ways of connecting wirelessly to recharge 722. The orb has a built-in energy source and ways of connecting wirelessly to recharger 720. In this configuration the orb communicates wirelessly 714 to the helmet.

The housing may be radio frequency shielded, such as by a Faraday shield, may provide an oxygen concentrator and CO₂ detector so that the local atmosphere around the user may be adjusted. For example, if a rise in the CO₂ content of the atmosphere around the user is detected by the CO₂ detector, O₂, from the O₂ concentrator, may be released reducing or diluting the CO₂ content locally around the user.

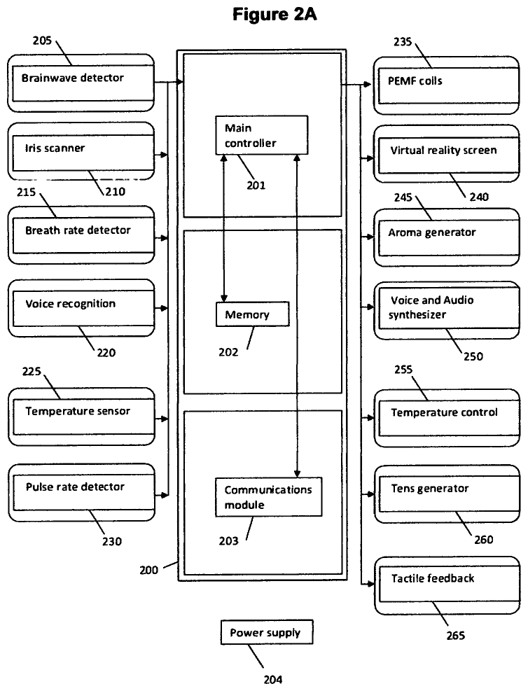

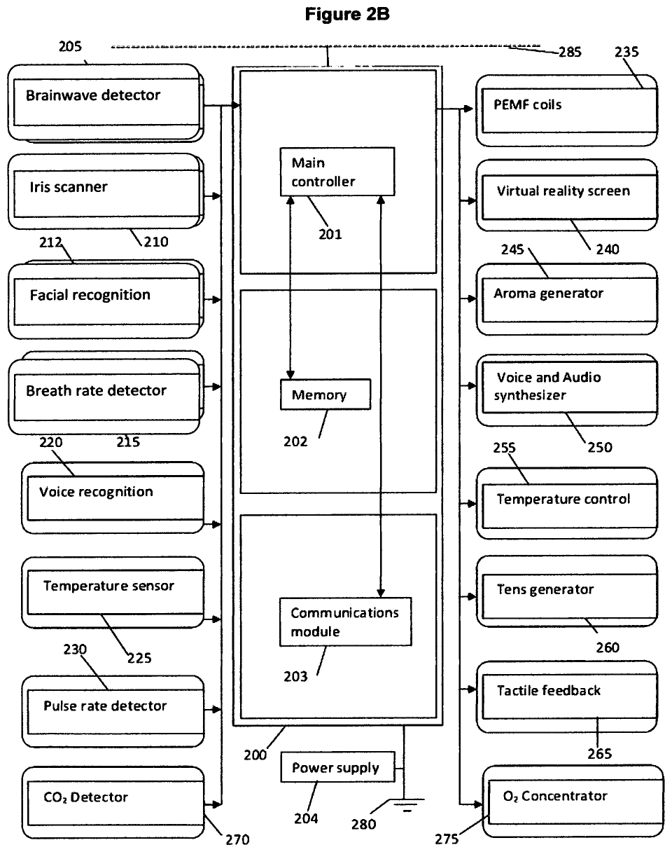

Any and all functions of the device shown in the block diagram in FIG. 2A-B may be integrated into a desired configuration. These functions include a brain wave detector/monitor 205, an iris scanner 210, a facial recognition system 212, a respiratory rate monitor 215, a voice recognition system 220, a temperature sensor 225 and heart or pulse rate monitor 230 acting as input circuits feeding signals to the System Controller 200, which is comprised of at least a main controller 201, a memory 202 and a communications module 203.

Carbon Dioxide (CO₂) detectors like the HT-2000 Portable 9999 ppm CO2 Meter Monitor Detector Gas Analyzer use Stable NDIR (Non-Dispersive Infrared) sensor for Carbon Dioxide concentration measurement and may be purchased commercially from a variety of providers including MXBAOHENG (China).

Brainwave monitor/detectors generally consist of an electrophysiological (EEG) monitoring system that measures cerebral electrical activity and may be purchased commercially from a variety of providers including Persyst (Solana Beach, Calif., USA), Emotiv (San Francisco, Calif., USA) and NR Sign (Vancouver, Canada). These work by measuring the voltage fluctuations from ionic current within the neurons of the brain. Brainwave detectors are most commonly used to diagnose epilepsy but are also used to diagnose insomnia, brain lesions, altered states of consciousness, and brain death. Brainwave detectors are usually non-invasive and require electrodes to be placed on the outside of the scalp for the duration of the test (usually 20-30 minutes). However, there are more invasive forms. Brainwave monitor/detector systems are digital and their signals are treated by computer. Machines are characterized principally by the number of channels it measures or reads. For example, the greater the number of channels, the more precisely it will locate the origin of an epileptic seizure.

Iris scanners measure the unique patterns in the colored circle of your eye to verify and authenticate your identity. Iris scanners work with a subject positioned about 3 inches to about 14 inches from the enrollment optional unit which is guided by a mirror, audio-assisted interactive interface to allow an auto-focus camera to take a digital video of the iris. A variety of iris scanners are available commercially and may be purchased from, for example, Crossmatch Technologies (Palm Beach Gardens, Fla., USA), Smartmatic (London, UK), and Griaule Biometrics (Campinas, Brazil). Individual images from the live video are captured using a frame grabber. The innovative algorithm of the iris recognition process analyzes the patterns in the iris that are visible between the pupil and sclera (white of the eye) and converts them into a 512-byt digital template. The iris is fully formed by 10 months of age and remain unchanged throughout an individual's lifetime. The pupils respond to more than changes in light, they show mental and emotional commotion. Researchers use pupil size changes, or pupillometry, to investigate a wide range of psychological phenomena. Pupillometry is a valuable tool for psychological research because our eyes provide a sensitive indicator of cognitive, emotional and sensory response.

Facial recognition systems can identify individuals as well as recognize and distinguish expressions such as sadness and anxiety. Noldus (Sacramento, Calif.) supplies FaceReader software for automatic facial expressions analysis.

Respiratory rate detector/monitor measures breathing rates by using audio sensors or pressure sensors with a filter to only detect breathing. A variety of respiratory rate detector/monitors are available commercially and may be purchased from, for example, Spire (San Francisco, Calif., USA) and Vandrico, Inc. (Vancouver, British Columbia, Canada, Brazil). These devices record the user's pulmonary ventilation. Breathing data is fed to algorithms that de-noise and classify it according to the way you normally breathe (e.g., what is calm for one individual may be different than calm for another individual). These algorithms use many characteristics of the respiratory waveform: inhalation/exhalation duration and slope, hold durations after inhale and exhale, inhalation/exhalation ratio, and consistency. When an individual is stressed, the body's ancient defense mechanisms are activated and become prepared for “fight or flight” mode—to run, to attack, or to do something that requires high physical activity. One thing that happens during this response is that breathing becomes rapid and shallow. The studies underlying the regulation of state of mind by respiration are summarized at http://en.wikipedia.org/wiki/Vagal_tone and http://en.wikipedia.org/wiki/Polyvagal_Theory—were spear-headed by Spire's scientific advisor, Dr. Stephen Porges.

Voice recognition allows recognition and translation of spoken language into text by computers. Voice recognition systems recognize the speaker, rather than what they are saying. Recognizing the speaker can simplify the task of translating in systems that have been trained on a specific person's voice or it can be used to authenticate or verify the identity of a speaker as part of a security process. A variety of voice recognition systems are available commercially and may be purchased from, for example, Olympus (Center Valley, Pa., USA), Baidu (Sunnyvale, Calif., USA), Apple (Cupertino, Calif., USA), Nuance (Burlington, Mass., USA), Microsoft (San Diego, Calif., USA) and Google (Mountain View, Calif., USA). To convert speech to on-screen text, a computer picks up on the vibrations in the air caused by the speaker's voice. The analog-to-digital converter (ADC) translates this analog wave into digital data that the computer can understand. The system then filters the digitized sound to remove unwanted noise and separates it into different bands of frequency. Then the signal is divided into small segments and the program then matches these segments to known phonemes (smallest element of language) in the appropriate language. These systems are commonly used in the healthcare setting for the medical documentation process.

Temperature sensor and control system measures the temperature at a determined area and use a thermal control system to adjust the temperature as desired. There are a wide variety of temperature measurement devices including thermometers, probes, resistance temperature detectors (RTDs), as well as non-contact detectors. A variety of temperature sensors and control systems are available commercially and may be purchased from, for example, Braintree Scientific, Inc. (Braintree, Mass., USA), TE Technology, Inc. (Traverse City, Mich., USA) and Minco (West Kelowna, British Columbia, Canada). To increase the temperature, some systems use far infrared radiation to penetrate deep into the body. Another method is using heating elements in flexible strips. To reduce temperature, petlier junctions can cool as well as heat.

Pulse rate detector/monitor is a personal monitoring device that measures a heart rate in real time or records the heart rate for later study. A variety of pulse rate detectors/monitors are available commercially and may be purchased from, for example, Santa Medical (Tustin, Calif., USA), Innovo (Elk Grove Village, Ill., USA), FaceLake (Lake Bluff; IL, USA) and Respironics (Murraysville, Pa., USA). The pulse sensor fits over a fingertip and works via pulse oximetry, a measurement technique that takes advantage of the fact that oxygenated and deoxygenated hemoglobin have different optical properties. With every heartbeat, there is a spike in arterial blood, which is detected as a change in the absorbance and/or reflectance of red and/or infrared light.

Heart rate detector/monitor measures the variation in the time interval between heartbeats. A variety of heart rate detectors/monitors are available commercially and may be purchased from, for example, NeuLog (Burlington, N.C., USA) and Mortara Burdick (Milwaukee, Wis., USA). When monitoring the intervals, either the heart rate at any point in time or the intervals between successive normal complexes are determined. The most common type of HRV is an electrocardiogram (ECG), which records the electrical activity of the heart. Small electrical impulses are created in the hearty by pacemaker cells. These impulses spread through the heart muscle and make it contract. ECG records these signals as they travel through the heart. Measuring the HRV reveals a wide range of information and the body and health of a patient.

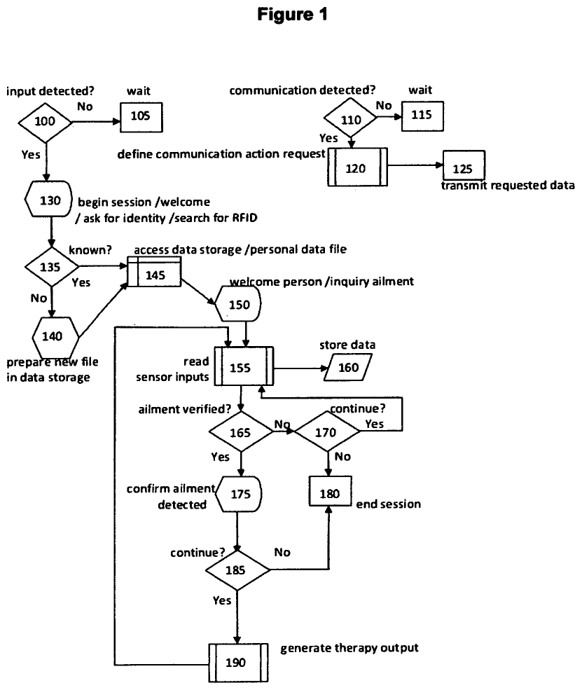

In all configurations the system controller 200 analyzes the data gathered by the input circuits, and per the system flow chart in FIG. 1, utilizes system outputs comprised of PEMF coils 235, a virtual reality screen 240, an aroma generator 245, a voice and audio synthesizer 250, a temperature control system 255, a TENS generator 260 and tactile feedback 265 to begin a treatment that will adjust the emotional state or mood of the individual. In general reducing an elevated or agitated state to a more normal relaxed state.

PEMF (pulsed electromagnetic field therapy) coils use electromagnetic fields to heal a variety of ailments. PEMF coils are available commercially and may be purchased from Curatronic (Petah Tikva, Israel). The United States Food and Drug Administration first accepted the use of PEMF therapy devices in the healing of non-union bone fractures in 1979, urinary incontinence and muscle stimulation in 1998, and depression and anxiety in 2006. Other countries around the world have accepted the use of PEMF for migraine headaches, recovery from trauma, degeneration, and even cancer. PEMF therapy increases energy levels in all cells of the body. The blood reflects these changes drastically in a short amount of time. For example, red blood cells become energized and white blood cells increase in size and mobility and move actively to catch bacteria and other pathogens.

Virtual reality screens or devices encompass technologies that are designed to provide visual, auditory and/or tactile stimulation to simulate a new reality. These devices are becoming more mainstream and affordable on the U.S. markets. They are also finding applications beyond the entertainment field to include medical and military applications. A variety of virtual reality screens or devices are available commercially and may be purchased from, for example, VRMADA (Madrid, Spain), Oculus (Los Angeles, Calif., USA), Microsoft (San Diego, Calif., USA), Google (Mountain View, Calif., USA), Samsung (Ridgefield Park, N.J., USA), and WorldViz (Santa Barbara, Calif., USA). One of the most attractive aspects of the future of virtual reality is the ability to simulate real life situations without the associated costs. For example, a surgeon obtaining real experience performing surgeries without placing a live patient in peril. Another example that is gaining traction in the market is a virtual reality for soldiers in training.

Aroma Generators are used with aroma oils or powders to produce powerful scents suitable for story telling or theming. A variety of aroma generators are available commercially and may be purchased from, for example, Experia (Buford, Ga., USA), Aromatech (Orlando, Fla., USA) and ScentSciences (Santa Clara, Calif., USA). Essential oils have been used for nearly 6,000 years, with the aim of improving a person's health or mood. A wide range of diverse smelling oils can be adiabatically dispersed or heated to emanate odors that trigger memories as can dry scent delivery systems. These devices can be programmed to emit a variety of aromas on digital cue. Inhaling essential oils stimulates the olfactory system, the part of the brain connected to smell, including the nose and the brain. Molecules that enter the nose or mouth pass to the lungs, and from there, to other parts of the body. As the molecules reach the brain, they affect limbic system, which is linked to the emotions, the heart rate, blood pressure, breathing, memory, stress, and hormone balance. In this way, aromas can have a subtle, yet holistic effect on the body. It has been shown to reduce anxiety, agitation, stress and depression.

Oxygen concentrators provide high concentration O₂ to the individual. Carbon dioxide levels above 350 ppm have been shown to dampen cognitive reasoning. Above 350 ppm the blood pH level shifts signaling the hemoglobin to carry less oxygen. The brain uses oxygen to function normally. Using oxygen to dilute the CO₂ available to the individual provides a dual benefit realized by reducing the CO₂ and increasing the oxygen the individual is breathing. Oxygen Concentrators are available commercially and may be purchased from, for example, Inogen (Goleta, Calif.) makers of the Inogen One portable oxygen concentrator.

Voice and audio synthesizers are the artificial production of human speech. A variety of voice and audio synthesizers are available commercially and may be purchased from, for example, Microsoft (San Diego, Calif., USA), Mattel (El Segundo, Calif., USA) and Apple (Cupertino, Calif., USA). A synthesizer takes the text as input and produces an audio stream as output. Speech synthesis is also referred to as text-to-speech (TTS). Text analysis uses natural language rules and analyzes a string of characters to determine where the words are and identifies grammatical details like functions and parts of speech. The sound generation takes this analysis and generates the appropriate sounds for the input text. Modem synthesizers use a database of sound segments built from recorded speech.

Transcranial stimulation is a noninvasive procedure that uses magnetic fields to stimulate nerve cells in the brain to improve symptoms of depression. A variety of transcranial stimulation devices are available commercially and may be purchased from, for example, MagVenture (Farum, Denmark) and Magstim (Whitland, UK). Transcranial stimulation is typically used when other treatments haven't been effective. An electromagnetic coil is placed against the patient's scalp near their forehead and painlessly delivers a magnetic pulse that stimulates nerve cells in the region of the brain involved in mood control and depression. The biology of why transcranial stimulation isn't exactly understood yet, however, the stimulation appears to affect how this part of the brain is working, which in turn seems to ease depression symptoms and improve mood.

Electric brain stimulation is a form of electrotherapy and technique used in research and clinical neurobiology to stimulate a neuron or neural network in the brain through the direct or indirect excitation of its cell membrane by using an electric current. Transcutaneous electrical nerve stimulation (TENS) is the use of electric current produced by a device to stimulate the nerves for therapeutic purposes. A variety of TENS generators are available commercially and may be purchased from, for example, Neurotech (Cumberland, R.I., USA). TENS generators work by sending stimulating pulses across the surface of the skin and along the nerve strands. The stimulating pulses help prevent pain signals from reaching the brain. TENS devices also help stimulate your body to produce higher levels of endorphins. When using a TENS device, electrodes are placed at specific sites on a user's body depending on the physical location of their pain. The current travels through the electrodes and into the skin stimulating specific nerve pathways to produce a massaging sensation that reduces the perception of pain.

Tactile feedback systems are systems that have been developed in order to provide augmentative sensory feedback for a number of medical applications. A variety of tactile feedback systems are available commercially and may be purchased from, for example, Intuitive Surgical, Inc. (Sunnyvale, Calif., USA). The key component of the system is a pneumatic balloon-based tactile display, which can be scaled and adapted for a variety of configurations, a commercial force sensor modified to fit the desired application. To date, this technology has been successfully applied to medical robotics, minimally invasive surgery, and rehabilitation medicine.

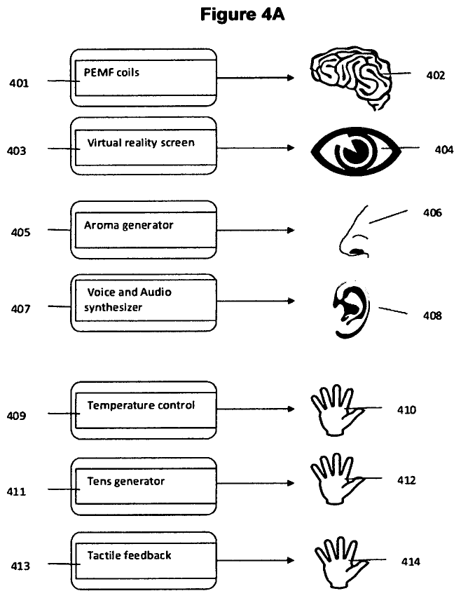

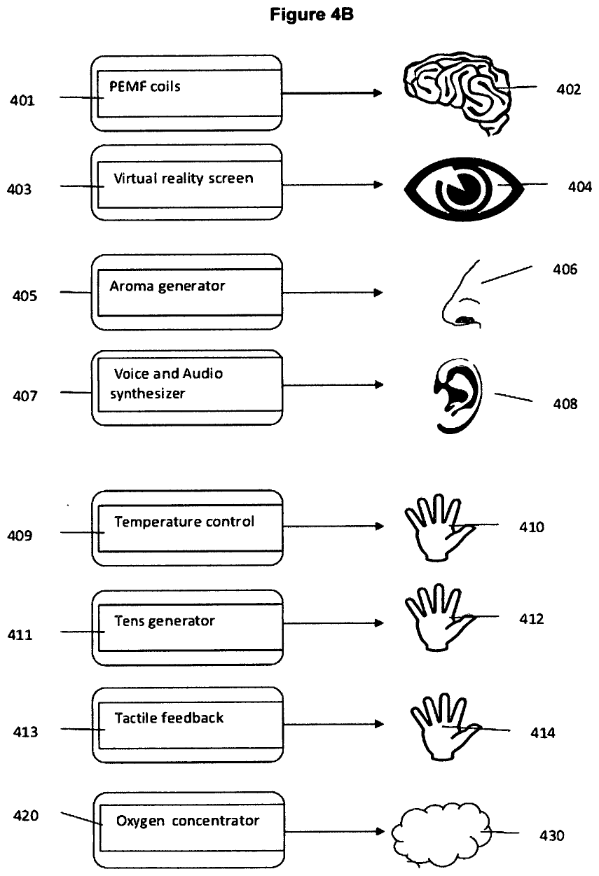

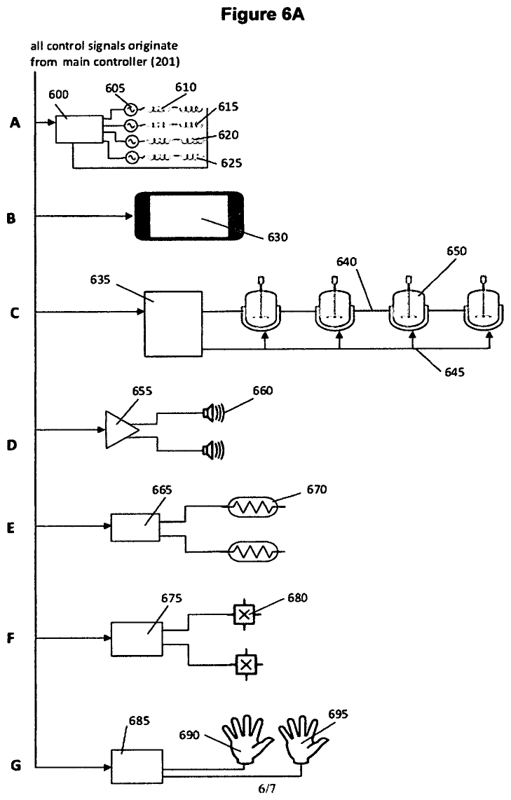

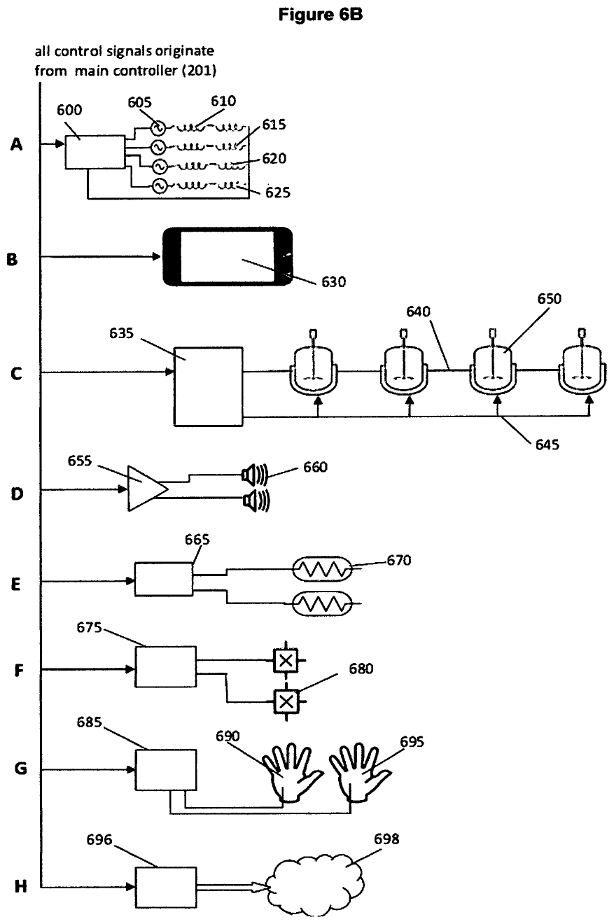

When using the helmet configured device, a person such as an astronaut will place the helmet over the head triggering one of the input circuits 100, which removes the unit from its wait state 105 and begins the session 130 (FIG. 1). The system controller 200 (FIG. 2) sends signals to the voice and audio synthesizer 407 (FIG. 4) that uses amplifier 655 to power speakers 660 (FIG. 6) so that audio commands and menus can be heard by the persons ears 408 (FIG. 4).

Simultaneously these audio commands and menus are displayed on virtual reality screen 403 (FIG. 4), which is integrated into the device 630 (FIG. 6), which can be seen and optically controlled by the user 404 (FIG. 4). The first instruction shown and vocalized is to hold the orb 716 (FIG. 7).

At the beginning of the session 130 (FIG. 1), the system controller communications module 203 (FIG. 2A-B) will attempt to read a local radio frequency identification (RFID) tag. If an RFID tag is recognized or known 135, the system will access data storage 145 (FIG. 1) from memory. 202 (FIG. 2). If the RFID tag is unidentified or unknown the system will prepare a new file in data storage 140 and store the personal data file 145 (FIG. 1).

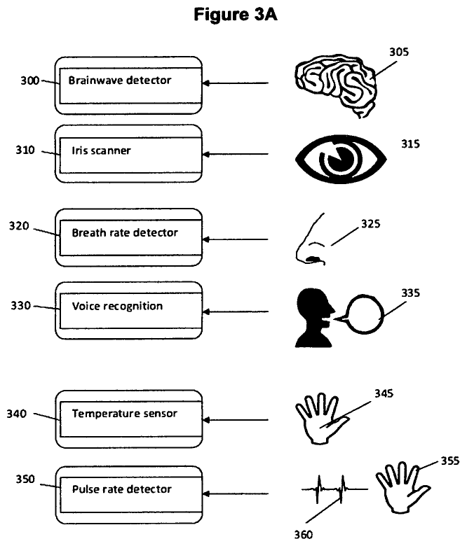

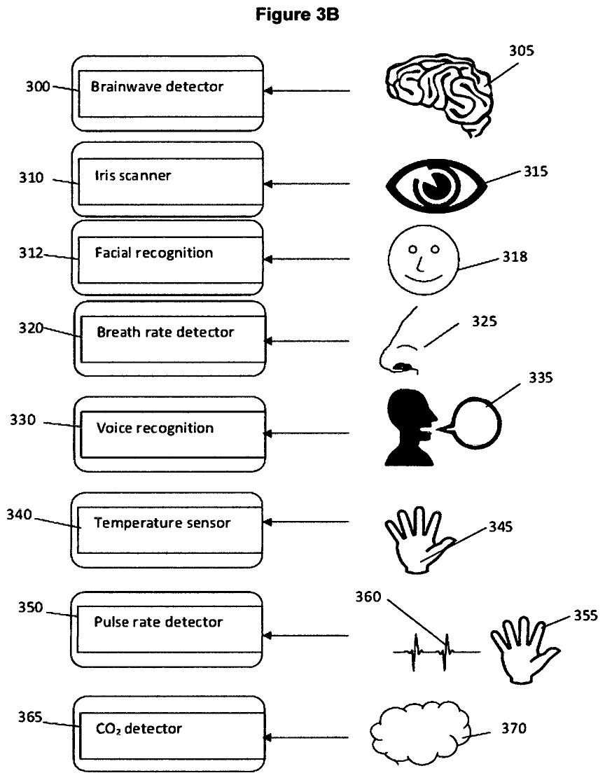

After the personal data file is either accessed or created the individual is welcomed and inquired about their condition, concern or ailment 150 (FIG. 1) both verbally and visually. The individual's voice commands 335 are voice recognized 330 (FIG. 3A-B) and used as inputs 220 into the system controller 200 (FIG. 2A-B). The system controller 200 (FIG. 2A-B) then begins to read all the sensor inputs 155 simultaneously storing all data 160 (FIG. 1).

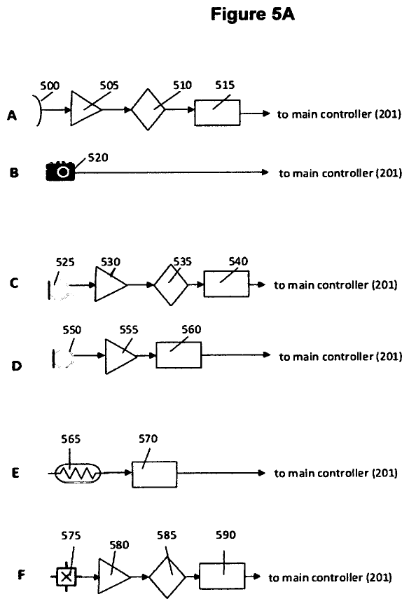

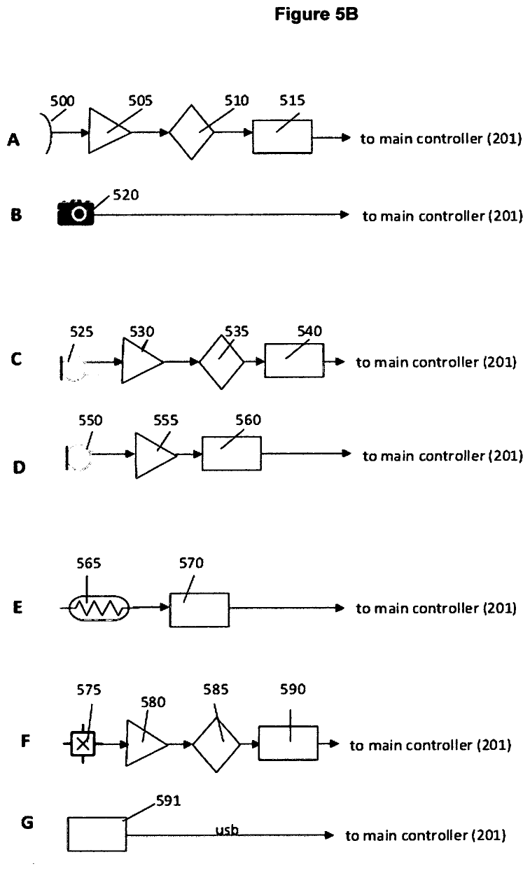

Alpha, beta and delta brain waves generated by the brain 305 may be detected by the brain wave detector/monitor 300 (FIG. 3A-B), which is comprised of antennas 500, amplifiers 505, bandpass filters 510, and analog to digital converters 515 (FIG. 5A-B).

The individual's eyes 315 (FIG. 3A-B) may be scanned by an iris scanner with the information collected by a digital camera 520 (FIG. 5A-B) and the information digitized 310 (FIG. 3A-B).

The sounds from respiratory activity (e.g., breathing) through the nose 325 may be detected by the respiratory rate detector/monitor 320 (FIG. 3A-B), which is comprised of a microphone 525, amplifier 530, bandpass filter 535 and analog to digital converter 540 (FIG. 5A-B).

The individual's temperature may be measured by thermal sensors placed in the hand receptors or indents on the orb 718 (FIG. 7) and programmed to measure the temperature of the hands 345. The temperature sensor 340 (FIG. 3A-B) is comprised the thermal sensor 565 and the analog to digital converter 570 (FIG. 5A-B).

The individual's heart or pulse rate 360 may be detected and monitored from the individual's hands 355 (FIG. 3A-B) as they are placed in the orb 716 (FIG. 7). The heart or pulse rate detector 350 (FIG. 3A-B) is comprised of an electrode 575 feeding into an amplifier 580, band pass filter 585, and analog-to-digital converter 590 (FIG. 5A-B).

Data from all the sensor inputs may be compared to baseline measurements for that given individual or known norms for a human having similar characteristics (e.g., sex, height, weight, age, race, origin and the like). If the issue of concern or ailment is verified 165 a message confirming the concern or ailment was detected 175 (FIG. 1) is communicated to the individual. The individual is then queried whether they want to continue with the prescribed treatment or treatments 185 as presented or if they wish to end the session 180 (FIG. 1). If the issue of concern or ailment could not be verified the individual is queried as to whether they want to continue or not 170. If they do, the process of reading the sensory inputs 155 is repeated. If they do not, the session is terminated 180 (FIG. 1). If the choice to continue 185 is made the device will generate pre-prescribed therapy outputs 190 (FIG. 1).

If the choice to continue 185 (FIG. 1) is made, then the CO₂ detector 591 (FIG. 5) will measure the atmosphere in housing 370 (FIG. 3A-B) and the system controller 200 (FIG. 2A-B) will control the oxygen concentrator 696 (FIG. 6) to reduce the CO₂ PPM atmosphere with increased O₂ PPM atmosphere in housing in housing 698.

If the prescribed treatment involves brain stimulation, the PEMF coils 401 may be focused into different regions of a brain 402 (FIG. 4A-B). Symmetrical left and right, forward and back coils 610, 615, 620, 625 that are driven by sine wave generators 605 create precisely placed (i.e., in the X, Y and Z axis) scalar waves when correctly timed by the signal generator 600 (FIG. 6).

If dynamic light therapy or visual therapy is prescribed, entertainment and prescribed visual stimulus may be perceived 404 on the virtual reality screen 403 (FIG. 4A-B) which is made of a monitor or smartphone 630 (FIG. 6).

If the prescribed treatment involves olfactory stimulus an aroma generator 405 (FIG. 4), which was a comprised of a controller 635, may supply compressed air 645 and control signals 640 to numerous aroma filled canisters or containers 650 (FIG. 6).

Sound and acoustic therapies may be applied to the individual if prescribed by a voice and audio synthesizer 407 (FIG. 4A-B) that utilizes an amplifier 655 driving stereo speakers 660 (FIG. 6).

If temperature adjustment is prescribed the temperature of the hands 410 may be adjusted by temperature controller 409 (FIG. 4A-B) that has left and right hand temperature pads 670 which are temperature controlled by Pelletier controller 665 (FIG. 6). Temperature adjustment for the body may be achieved in other configurations wherein the body of the individual is within a partial or complete chamber. In this configuration, the chamber temperature may be regulated as prescribed.

If tactile stimulation is prescribed, there may be electrodes in the orb that connect the individual's hand 412 to TENS generator 411 (FIG. 4A-B). The TENS generator has electrodes 680, one for each hand, that receives signals from the TENS signal generator 675 (FIG. 6).

Alternatively, the individual's hands 414 (FIG. 4A-B) placed on the orb 716 (FIG. 7) may be pneumatically massaged with individually adjustable finger pressures and vibrations generated by a tactical feedback unit 413, which uses pneumatic and piezoelectric controls (FIG. 4A-B). The main controller 201 (FIG. 2A-B) generates the output signals that direct the tactical feedback generator 685 to properly control the transducers in the orbs hand wells 690 and 695 (FIG. 6).

The present invention also monitors the individual's heart or pulse rate 360 variability and respiratory activity (e.g., sounds of breathing) 325 to show the real time reaction to treatments as they are administered.

If a choice is made not to continue the session, the session will end 180 and all sensor inputs that were read during the session are stored in memory 160 that will be available for access 145 during the next session (FIG. 1).

If communication is required during the session or after, a communication request is detected 110 (FIG. 1) and the request will activate the communications module 203 (FIG. 2A-B). The communications module will define the communication action request 120 and transmit or receive data as requested 125 (FIG. 1).

The entire system is powered by a power supply 204. Power to wirelessly recharge the helmet 700 and orbs 716 is delivered through transmitter 722. This occurs whenever the helmet 700 is placed on or near to the transmitter 722 (FIG. 7).

While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

It is an advantage of the present invention to provide individuals with an automated emotional state adjustment system for diagnosis and treatment of unwanted emotional states or moods, which uses digital technology for: rapid sampling of multiple detector and sensors; analyzing the individuals emotional state or mood; communicating information to the individual user and/or to remote locations; and providing various therapies appropriate for the symptoms detected.

It is a further object of the present invention to provide a method for individuals to privately and safely express their emotions and communicate their feelings while simultaneously being diagnosed for symptoms and receive treatments to reduce or alleviate unwanted emotional states.

In an exemplary embodiment, the device of the present invention provides a method to systematically identify and diagnose an individual while simultaneously measuring the biologic reactions of the individual to prescribed therapies recommended to relieve undesirable emotional states such as stress, anxiety and the like.

I claim:

1. A device for adjusting an individual's emotional state or mood comprising:
a housing;
a plurality of detectors or sensors for obtaining information on the physical characteristics of said individual affixed within said housing;
a plurality of sensory stimulus generators for providing sensory stimulus to said individual connected to said housing;
a controller that receives information from said plurality of detectors or sensors and analyzes said information to determine a physical treatment, wherein said plurality of sensory stimulus generators are connected to said controller and wherein said physical treatment is provided by one or more of said plurality of sensory stimulus generators, wherein said controller is affixed within said housing;
a memory connected to said controller for storing said information received by said plurality of detectors or sensors, said analyzes performed by said controller and said physical treatment as applied by said sensory stimulus generators as directed by said controller, wherein said memory is affixed within said housing;
a user interface connected to said controller to allow said individual to communicate with said controller, wherein said user interface is connected to said housing;
a medical practitioner interface connected to said controller for accessing information stored in said memory or for providing instructions to said controller or to interface directly with said individual; and
a power supply connected to said controller.

2. The device according to claim 1, wherein the device further comprises a radio frequency shielded housing and wherein the power supply is earth grounded.

3. The device according to claim 1, wherein said plurality of detectors or sensors are at least three of said detectors or sensors, wherein said detectors or sensors are a combination of a brain wave monitor, an iris scanner, a respiratory monitor, a temperature sensor, a heart rate or pulse rate monitor, a facial recognition system or a voice recognition system.

4. The device according to claim 1, wherein said plurality of sensory stimulus generators are at least two of said sensory stimulus generators, wherein said sensory stimulus generators are a combination of a PEMF coils, a virtual reality image screen, an aroma generator, a temperature control, a tens generator or a tactile feedback system.

5. The device according to claim 1, wherein said user interface is a written communication system or a verbal communication system.

6. The device according to claim 5, further comprising a iris detection system, wherein said iris detection system is utilized to provide the written and/or verbal communication systems.

7. The device according to claim 1, wherein said power supply is a rechargeable battery replaceably affixed within said housing.

8. The device according to claim 1, wherein said housing is a helmet, a desk top mounted device, a free standing device, a partial body chamber, a full body chamber.

9. The device according to claim 1, wherein said medical practitioner interface is connected to said controller via a wireless connection.

10. A method of adjusting an individual's emotional state or mood comprising the steps of:
having said individual desiring adjustment of their emotional state or mood interface with a device for adjusting an individual's emotional state or mood, wherein said device comprises: a housing;
a plurality of detectors or sensors for obtaining information on the physical characteristics of said individual affixed within said housing;
a plurality of sensory stimulus generators for providing sensory stimulus to said individual connected to said housing;
a controller that receives information from said plurality of detectors or sensors and analyzes said information to determine a physical treatment, wherein said plurality of sensory stimulus generators are connected to said controller and wherein said physical treatment is provided by one or more of said plurality of sensory stimulus generators, wherein said controller is affixed within said housing;
a memory connected to said controller for storing said information received by said plurality of detectors or sensors, said analyzes performed by said controller and said physical treatment as applied by said sensory stimulus generators as directed by said controller, wherein said memory is affixed within said housing;
a user interface connected to said controller to allow said individual to communicate with said controller, wherein said user interface is connected to said housing; and
a power supply connected to said controller obtaining information on the physical characteristics of said individual from said plurality of detectors or sensors;
analyzing said information to determine a physical treatment for said individual;
instituting said treatment by providing sensory stimulus through said sensory stimulus generators, thereby adjusting the emotional state or mood of said individual; and
storing said information of said physical characteristics, analyses and physical treatment of said individual.

11. The method according to claim 10, wherein the device further comprises a radio frequency shielded housing and wherein the power supply is earth grounded.

12. The method according to claim 10, wherein said plurality of detectors or sensors are at least three of said detectors or sensors, wherein said detectors or sensors are a combination of a brain wave detector, an iris scanner, a facial recognition system a breath rate detector, a temperature sensor, a heart rate, a voice recognition system or pulse detector.

13. The method according to claim 10, wherein said plurality of sensory stimulus generators are at least two of said sensory stimulus generators, wherein said sensory stimulus generators are a combination of a pemf coils, a virtual reality image screen, an aroma generator, a temperature control, a tens generator or a tactile feedback system.

14. The method according to claim 10, wherein said user interface is a written communication system or a verbal communication system.

15. The method according to claim 14, wherein the device further comprising a iris detection system, wherein said iris detection system is utilized to provide the written and/or verbal communication systems.

16. The method according to claim 10, wherein said power supply is a rechargeable battery replaceably affixed within said housing.

17. The method according to claim 10, wherein said housing is a helmet, a desk top mounted device, a free standing device, a partial body chamber, a full body chamber.

18. A method of adjusting an individual's emotional state or mood comprising the steps of:
having said individual desiring adjustment of their emotional state or mood interface with a device for adjusting an individual's emotional state or mood, wherein said device comprises:
an atmospherically controlled housing;
a plurality of detectors or sensors for obtaining information on the physical characteristics of said individual affixed within said atmospherically controlled housing;
a plurality of sensory stimulus generators for providing sensory stimulus to said individual connected to said atmospherically controlled housing;
a controller that receives information from said plurality of detectors or sensors and analyzes said information to determine a physical treatment, wherein said plurality of sensory stimulus generators are connected to said controller and wherein said physical treatment is provided by one or more of said plurality of sensory stimulus generators, wherein said controller is affixed within said atmospherically controlled housing;
a memory connected to said controller for storing said information received by said plurality of detectors or sensors, said analyzes performed by said controller and said physical treatment as applied by said sensory stimulus generators as directed by said controller, wherein said memory is affixed within said atmospherically controlled housing;
a user interface connected to said controller to allow said individual to communicate with said controller, wherein said user interface is connected to said atmospherically controlled housing; and
a housing, wherein said housing is radio frequency shielded and atmospherically controlled, and
a earth grounded power supply connected to said controller
obtaining information on the physical characteristics of said individual from said plurality of detectors or sensors;
analyzing said information to determine a physical treatment for said individual;
instituting said treatment by providing sensory stimulus through said sensory stimulus generators, thereby adjusting the emotional state or mood of said individual; and
storing said information of said physical characteristics, analyses and physical treatment of said individual.

19. The method according to claim 18, wherein said device further comprises a carbon dioxide detector.

20. The method according to claim 18, wherein said device further comprises an oxygen concentrator.
